(19) United States
(12) Patent Application Publication
(10) Pub. No.: US 2007/0276004 A1
(43) Pub. Date: Nov. 29, 2007

(54) Pharmaceutical Composition Comprising an Immunosuppressant for Use in the Treatment of Skin Diseases

(76) Inventors: Stefan Hirsch, Lorrach (DE); Friedrich Karl Mayer, Oberwil (CH); Carle Paul, Mulhouse (FR); Nabila Sekkat, Basel (CH); Carl-Martin Weiss, Lorrach-Haagen (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/550,357

(22) PCT Filed: Apr. 2, 2004
(86) PCT No.: PCT/EP04/03513

§ 371 Date: Feb. 23, 2007

(30) Foreign Application Priority Data
Apr. 4, 2003 (GB) 0307866.4

(51) Int. Cl. A61K 31/445; A61P 17/00

(52) U.S. Cl. 514/330

(57) ABSTRACT

Synergistic combinations of a macrolide T-cell immunomodulator or immunosuppressant such as 33-epichloro-33-desoxyascomycin and an emollient such as dimethicone, glycerol or isostearyl isostearate are provided, which are useful in particular in the treatment of dermatological or mucosal diseases such as dry skin or atopic or contact dermatitis.

The invention relates to pharmaceutical compositions, for use in particular in the treatment of skin diseases. It concerns a pharmaceutical composition comprising a macrolide T-cell immunomodulator or immunosuppressant and an emollient.

It has now been found that, surprisingly, macrolide T-cell immunomodulators and immunosuppressants, when used in combination or association with emollients, act synergistically, resulting in a potentiation of pharmacological activity, such that effective beneficial, especially anti-dermatitis activity is seen upon co-administration at dosages which would be well below the effective dosages administered individually.

The invention thus concerns novel pharmaceutical compositions comprising a macrolide T-cell immunomodulator or immunosuppressant in association or combination with an emollient, hereinafter briefly named “the compositions of the invention”.

A macrolide T-cell immunomodulator or immunosuppressant is to be understood herein as being a T-cell immunomodulator or T-cell immunosuppressant which has a macrocyclic compound structure including a lactone or lactam moiety. While it preferably has at least some T-cell immunomodulating or immunosuppressant activity, it may also exhibit concomitantly or predominantly further pharmaceutical properties, such as anti-inflammatory activity.

An emollient is to be understood herein as being an agent which softens or soothes the skin, or soothes an irritated internal surface.

It should be appreciated that the present invention does not contemplate merely the inclusion of an emollient as a minor excipient in a pharmaceutical composition comprising a macrolide T-cell immunomodulator or immunosuppressant in order to e.g. improve the compatibility of the composition as such with e.g. human skin. More comprehensively, it is contemplated herewith to involve emollients as active agents in their own right, whereby “active” should be understood as relating not only to pharmacological activity, but also activity as regards cosmetic aspects, such as the appearance or brittleness of skin.

The amount of emollient to be used or included with the compositions of the invention is thus normally substantially more than commonly used in pharmaceutical compositions, or is administered separately from the macrolide. It is e.g. from about 10% to about 5000%, preferably from about 20% to about 1000%, more preferably from about 100% to about 500% w/w of the amount of macrolide in the composition.

The compositions of the invention may thus be viewed also as health care or personal care products incorporating at least one pharmaceutically active component, or as so-named “cosmeceuticals”.

The compositions of the invention may be adapted for systemic use as regards the immunomodulator or immunosuppressant component, e.g. oral or intravenous, or for topical use for both components; preferably they are adapted for topical use. They are useful for the known indications of the particular active agents incorporated therein. They are particularly indicated for use in dermatological or mucosal diseases, e.g. dermatological or mucosal diseases which have an inflammatory component or involve inflammatory complications, such as dry skin or atopic or contact dermatitis.

The composition resulting from the combination is e.g. a medicated emollient, appropriately presented, e.g. as a poultice or a cataplasm.

A suitable macrolide T-cell immunomodulator or immunosuppressant is for example an FKBP12-binding calcineurin inhibitor or mitogen-activated kinase modulator or inhibitor, in particular an asco- or rapamycin. It preferably is an ascomycin. While the macrolide preferably has at least some calcineurin- or mitogen-activated kinase modulating or inhibiting activity, it may also exhibit concomitantly or predominantly further pharmaceutical properties, such as antiinflammatory activity. It preferably is a compound, e.g. an ascomycin, having rather long-acting activity relatively to other members of the same structural class, e.g. it is metabolically degraded slowly to inactive products.

An asco- or rapamycin is to be understood as asco- or rapamycin as such, or a derivative thereof. An asco- or rapamycin derivative is to be understood as being an antagonist, agonist or analogue of the parent compound which retains the basic structure and modulates at least one of the biological, for example immunological properties of the parent compound.

An “anti-inflammatory ascomycin derivative” is defined herein as an ascomycin derivative that exhibits pronounced anti-inflammatory activity in e.g. animal models of allergic contact dermatitis but has only low potency in suppressing systemic immune response, namely, which has a minimum effective dose (MED) of up to a concentration of about 0.04% w/v in the murine model of allergic contact dermatitis upon topical administration, while its potency is at least 10 times lower than for tacrolimus (MED 14 mg/kg) in the rat model of allogeneic kidney transplantation upon oral administration (Meingassner, J. G. et al., Br. J. Dermatol. 137 [1997] 568-579; Stuetz, A. Seminars in Cutaneous Medicine and Surgery 20 [2001] 233-241). Such compounds are preferably lipophilic.

Suitable ascomycins are e.g. as described in EP 184162, EP 315978, EP 323042, EP 423714, EP 427680, EP 465426, EP 474126, WO 91/13889, WO 91/19495, EP 484936, EP 523088, EP 532089, EP 569337, EP 626385, WO 93/5059 and WO 97/8182; in particular:
ascomycin;
tacrolimus (FK506; Prograf^{R});
imidazolylmethoxyascomycin (WO 97/8182 in Example 1 and as compound of formula I);
32-O-(1-hydroxyethylindol-5-yl)ascomycin (L-732531) (Transplantation 65 [1998] 10-18, 18-26, on page 11, FIG. 1; and
(32-desoxy-32-epi-N1-tetrazolyl)ascomycin (ABT-281) (J.Invest.Dermatol. 12 [1999] 729-738, on page 730, FIG. 1); preferably:
{1R,5Z,9S,12S-[1E-(1R,3R,4R)],13R,14S,17R,18E,21S,23S,24R,25S,27R}-17-ethyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo[22.3.1.0(4,9)]octacos-5,18-diene-2,3,10,16-tetraone (Example 8 in EP 626385),
hereinafter referred to as “5,6-dehydroascomycin”;
{1E-(1R,3R,4R)]1R,4S,5R,6S,9R,10E,13S,15S,16R,17S,19S,20S}-9-ethyl-6,16,20-trihydroxy-4-[2-(4-hydroxy-3-methoxycyclohexyl)-1-methylvinyl]-15,17-dimethoxy-5,11,13,19-tetramethyl-3-oxa-22-azatricyclo[18.6.1.0(1,22)]heptacos-10-ene-2,8,21,27-tetraone (Examples 6d and 71 in EP 569337), hereinafter referred to as “ASD 732”; and especially
pimecrolimus (INN recommended) (ASM981; Elidel™), i.e. {[1E-(1R,3R,4S)]1R,9S,12S, 13R,14S,17R,18E,21S,23S,24R,25S,27R}-12-[2-(4-chloro-3-methoxycyclohexyl)-1-methylvinyl]-17-ethyl-1,14-dihydroxy-23,25-dimethoxy-13,19,21,27-tetramethyl-11,28, dioxa-4-azatricyclo[22.3.1.0(4,9)]octacos-18-ene-2,3,10,16-tetraone, of formula I 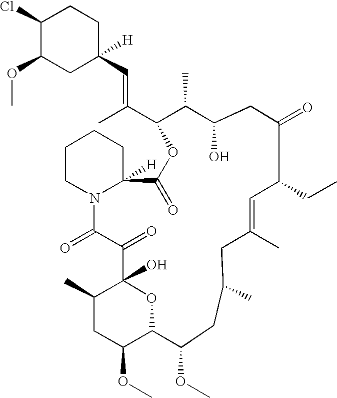
   (Example 66a in EP 427680),
   hereinafter also referred to as “33-epichloro-33-desoxyascomycin”.

Suitable anti-inflammatory ascomycin derivatives are e.g.: (32-desoxy-32-epi-N1-tetrazolyl)ascomycin (ABT-281); 5,6-dehydroascomycin; ASD 732; and pimecrolimus.

Suitable rapamycins are e.g. as described in U.S. Pat. No. 3,929,992, WO 94/9010 and U.S. Pat. No. 5,258,389, preferably sirolimus (rapamycin; Rapamune^{R}) and everolimus (RAD001; Certican^{R}).

A particularly preferred macrolide T-cell immunomodulator or immunosuppressant is pimecrolimus; it is in free form unless specified otherwise.

A suitable emollient is for example one-phase mineral oil (petrolatum), or mineral oil as a two-phase system, either as an oil-in-water or a water-in-oil emulsion, or as a lotion; it is e.g. a silicone such as dimethicone; glycerine; or vaseline. The system may be of low or high viscosity. It may form a hydrophobic protective film on the skin, as with e.g. a silicone such as dimethicone, or paraffin or petrolatum (vaseline). A humectant may be added as appropriate, e.g. glycerol; or an emollient which has semi-occlusive properties may be used, such as a fatty acid or a fatty acid ester, e.g. isostearyl isostearate. Preferred emollients are dimethicone, glycerol and isostearyl isostearate.

Emollients may thus be e.g. fatty alcohols, hydrocarbons, triglycerides, waxes, esters, silicone oils and lanolin containing products. Fatty alcohols are e.g. cetyl alcohol, octyldodecanol, stearyl alcohol and oleyl alcohol. Hydrocarbons include mineral oil, petrolatum, paraffin, squalene, polybutene, polyisobuten, hydrogenated polyisobutene, cerisin and polyethylene. Triglycerides are e.g. castor oil, caprylic/capric triglyceride, hydrogenated vegetable oil, sweet almond oil, wheat germ oil, sesame oil, hydrogenated cottonseed oil, coconut oil, wheat germ glycerides, avocado oil, corn oil, trilaurin, hydrogenated castor oil, shea butter, cocoa butter, soybean oil, mink oil, sunflower oil, safflower oil, macadamia nut oil, olive oil, apricot kernel oil, hazelnut oil and borage oil. Waxes include e.g. carnauba wax, beeswax, cadelilla wax paraffin, Japan wax, microcrystalline wax, jojoba oil, cetyl esters wax, and synthetic jojoba oil. Esters include e.g. isopropyl myristate, isopropyl palmitate, octyl palmitate, isopropyl linoleate, C₁₂₋₁₅ alcohol benzoates, cetyl palmitate, myristyl myristate, myristyl lactate, cetyl acetate, propylene glycol dicaprylate/caprate, decyl oleate, stearyl heptanoate, diisostearyl malate, octyl hydroxystearate and isopropyl isostearate. Silicone oils are e.g. dimethicone (dimethyl polysiloxane) and cyclomethicone. Lanolin containing products are e.g. lanolin, lanolin oil, isopropyl lanolate, acetylated lanolin alcohol, acetylated lanolin, hydroxylated lanolin, hydrogenated lanolin and lanolin wax.

Personal care products are e.g. shampoos, hair conditioners, combination shampoo/conditioner, shower gels, soaps, hair styling products, hair colorants, deodorants, antiperspirants and moisturizing lotions. The compositions of the invention may comprise, in addition, further active components which provide benefit to the hair or skin, e.g. moisturizing agents, antiperspirants, anti-bacterials, cleaning agents, hair conditioning agents, hair styling agents, anti-dandruff agents, hair growth promoters, hair dyes and pigments, soaps and perfimes.

The compositions of the invention may be e.g. creamy, of the “light” or “rich” type, or greasy, or containing urea. Further components may be selected from e.g. almond oil, cacao butter, castor oil, decyl oleate, triglyceride, cetostearyl ethylhexanoate, stearyl heptanoate or caprylate, diisopropyl adipate, tri-isononanoin, polyethyleneglycol-40 butyloctanol and trideceth-9, polyethyleneglycol-5-ethylhexanoate.

Subgroups of compositions of the invention comprise a macrolide T-cell immunomodulator or immunosuppressant, preferably an anti-inflammatory ascomycin derivative as defined above, especially pimecrolimus, in combination or association with an emollient other than the following emollients singly or collectively in any number:
glycerine; and/or
a fatty acid ester; and/or
a silicone; and/or
dimethicone; and/or
a fatty acid; and/or
petrolatum.

In a further subgroup of compositions of the invention the macrolide T-cell immunomodulator or immunosuppressant is other than tacrolimus; in a further subgroup it is other than tacrolimus and sirolimus.

Preferred for use in the treatment of conditions where inflammation is involved are compositions of the invention wherein one or both components possess some degree of inherent anti-inflammatory activity. Particularly preferred are compositions comprising an ascomycin, preferably an anti-inflammatory ascomycin derivative, especially pimecrolimus, in combination or association with an emollient; more especially pimecrolimus in combination or association with dimethicone, glycerol or isostearyl isostearate. The inflammatory condition is e.g. dry skin or atopic or contact dermatitis.

Pimecrolimus being anti-inflammatory and having excellent skin penetration but only minimal skin permeation properties, it is not having significant systemic side effects when applied topically on skin, and the soothing effect of emollients complements its anti-inflammatory action on inflamed skin. “Treatmenf” as used herein refers in particular to use for preferably alleviating an existing condition, namely curative treatment, although the invention also contemplates prophylactic use in conditions where a high probablity of inflammation exists.

Synergy is e.g. calculated as described in Berenbaum, Clin. Exp. Immunol. 28 (1977) 1, using an interaction term to correct for differences in mechanism between the two drugs, as described in Chou et al., Transpl. Proc. 26 (1994) 3043. The index of synergy is calculated as:

$$\frac{dose of A}{{A}_{E}} + \frac{dose of B}{{B}_{E}} + \frac{( dose of A ) ⨯ ( dose of B )}{{A}_{E} ⨯ {B}_{E}}$$

in which the doses of the compounds A and B represent those used in a particular combination, and A_{E} and B_{E} are the individual doses of A and B respectively giving the same effect. If the result is less than 1, there is synergy, if the result is 1, the effect is additive; if the result is greater than 1, A and B are antagonistic. By plotting an isobologram of dose of A/A_{E} vs. dose of B/B_{E} the combination of maximum synergy can be determined. The synergistic ratio expressed in terms of the ratio by weight of the two compositions at synergistic amounts along the isobologram, especially at or near the point of maximum synergy, can then be used to determine formulations containing an optimally synergistic ratio of the two compounds.

Activity may e.g. be determined in known assay models for testing the activity of the individual components of the compositions.

The invention also provides products and methods for co-administration of a macrolide T-cell immunomodulator or immunosuppressant, e.g. 33-epichloro-33-desoxy-ascomycin or 5,6-dehydroascomycin, and an emollient, e.g. dimethicone, at synergistically effective dosages, e.g.:
a method of treatment or prevention of a dermatological or mucosal disease such as dry skin or atopic or contact dermatitis in a subject suffering from or at risk for such condition, comprising co-administering synergistically effective amounts of a composition of the invention;
the use of a macrolide T-cell immunomodulator or immunosuppressant in the manufacture of a medicament for co-administration in synergistically effective amounts with an emollient;
the use of an emollient in the manufacture of a medicament for co-administration in synergistically effective amounts with a macrolide T-cell immunomodulator or immunosuppressant;
a kit of parts comprising a macrolide T-cell immunomodulator or immunosuppressant and an emollient in separate unit dosage forms, preferably wherein the unit dosage forms are suitable for administration of the component compounds in synergistically effective amounts, together with instruction for use, optionally with further means for facilitating compliance with the administration of the component compounds, e.g. a label or drawings;
the use of a macrolide T-cell immunomodulator or immunosuppressant in the manufacture of a pharmaceutical kit which is to be used for facilitating co-administration with an emollient;
the use of an emollient in the manufacture of a pharmaceutical kit which is to be used for facilitating co-administration with a macrolide T-cell immunomodulator or immunosuppressant;
a macrolide T-cell immunomodulator or immunosuppressant and an emollient as a combined pharmaceutical preparation for simultaneous, separate or sequential use, preferably in synergistically effective amounts, e.g. for the treatment or prevention of a dermatological or mucosal disease such as dry skin or atopic or contact dermatitis;
a pharmaceutical composition comprising a macrolide T-cell immunomodulator or immunosuppressant in combination or association with an emollient, e.g. in synergistically effective amounts, together with at least one pharmaceutically acceptable diluent or carrier, e.g. for use in treatment or prevention of a dermatological or mucosal disease such as dry slin or atopic or contact dermatitis; and
a process for the preparation of a composition of the invention comprising mixing a macrolide T-cell immunomodulator or immunosuppressant and an emollient, in combination or association with at least one pharmaceutically acceptable diluent or carrier.

By “synergistically effective amounts” is meant an amount of macrolide T-cell immunomodulator or immunosuppressant and an amount of emollient which are individually below their respective effective dosages for a relevant indication, but which are pharmaceutically active on co-administration, e.g. in a synergistic ratio, for example as calculated above. Furthermore, “synergistically effective amounts” may mean an amount of macrolide T-cell immunomodulator or immunosuppressant and an amount of emollient which are individually equal to their respective effective dosages for a relevant indication, and which result in a more than additive effect.

The molar amount of macrolide T-cell immunomodulator or immunosuppressant present is from roughly similar to, to significantly less than the amount of emollient, preferably half as much or less. Synergistic ratios of macrolide T-cell immunomodulator or immunosuppressant to emollient by weight are thus suitably from about 10:1 to about 1:50, preferably from about 5:1 to about 1:20, most preferably from about 1:1 to about 1:15, e.g. about 1:12.

The compositions of the invention can be administered as a free combination, or can be formulated into a fixed combination, which greatly enhances the convenience for the patient.

Absolute dosages of the compounds will vary depending on a number of factors, e.g. the individual, the route of administration, the desired duration, the rate of release of the active agent and the nature and severity of the condition to be treated. For example, the amount of active agents required and the release rate thereof may be determined on the basis of known in vitro and in vivo techniques, determining how long a particular active agent concentration in the blood plasma remains at an acceptable level for a therapeutic effect For example, in prevention and treatment of a dermatological or mucosal disease such as dry skin or atopic or contact dermatitis, an initial dosage of about 2-3 times the maintenance dosage is suitably administered, followed by a daily dosage of about 2-3 times the maintenance dosage for a period of from one to two weeks, and subsequently the dose is gradually tapered down at a rate of about 5% per week to reach the maintenance dosage. In general, synergistically effective amounts of 33-epichloro-33-desoxyascomycin and dimethicone on oral administration for use in prevention and treatment of dry skin or atopic or contact dermatitis in larger animals, e.g. man, are amounts of pimecrolimus of up to about 2 mg/kg/day, e.g. from about 0.01 mg/kg/day to about 2 mg/kg/day, preferably about 0.5 mg/kg/day, in combination or co-administration with amounts of dimethicone of up to about 50 mg/kg/day, e.g. from about 0.25 mg/kg/day to about 50 mg/kg/day, preferably about 2.5 mg/kg/day, in a synergistic ratio, as described. Suitable unit dosage forms for oral co-administration of these compounds thus may contain on the order of from about 0.5 mg to about 100 mg, preferably about 3 mg to about 30 mg of 33-epichloro-33-desoxyascomycin, and from about 10 mg to about 3000 mg, preferably about 50 mg to about 500 mg of dimethicone. The daily dosage for oral administration is preferably taken in a single dose, but may be spread out over two, three or four dosages per day. For i.v. administration, the effective dosage is lower than that required for oral administration, e.g. about one fifth the oral dosage.

By “co-administration” is meant administration of the components of the compositions of the invention together or at substantially the same time, e.g. within fifteen minutes or less, either in the same vehicle or in separate vehicles, so that upon oral administration, for example, both compounds are present simultaneously in the gastrointestinal tract. However, upon topical application, administration of the components may also be separated by a time interval of at least several hours, e.g. 6 hours or 12 hours. Preferably, the compounds are administered as a fixed combination, preferably topically.

The compositions of the invention include compositions suitable for administration by any conventional route, in particular compositions suitable for administration either enterally, for example, orally, e.g. in the form of solutions for drinking, tablets or capsules, or parenterally, e.g. in the form of injectable solutions or suspensions; or topically, e.g. for the treatment of inflammatory conditions of the skin or mucosae, e.g. in the form of a dermal crearn, ointment, ear drops, mousse, shampoo, solution, lotion, gel, emulgel or like preparation, e.g. in a concentration of from about 0.1% to about 2%, preferably about 1% by weight of each component, especially in combination or association with penetration enhancing agents, as well as for application to the eye, e.g. in the form of an ocular cream, gel or eye-drop preparation, for treatment of inflammatory conditions of the lungs and airways, e.g. in the form of inhalable compositions, and for mucosal application, e.g. in the form of vaginal tablets.

The compositions of the invention are suitably emulsions, microemulsions, emulsion preconcentrates or microemulsion preconcentrates, or solid dispersions, especially water-in-oil microemulsion preconcentrates or oil-in-water microemulsions, comprising the macrolide T-cell immunomodulator or immunosuppressant and the emollient in a synergistic ratio.

The compositions of the invention can be prepared in conventional manner, e.g. by mixing a macrolide T-cell immunomodulator or immunosuppressant and an emollient, in combination or association with at least one pharmaceutically acceptable diluent or carrier.

The active agent components may be in free form or pharmaceutically acceptable salt form as appropriate.

While the invention primarily contemplates combination or association ofjust two pharmaceutically and/or cosmetically active components, it does not exclude the presence of further pharmaceutically and/or cosmetically active agents, e.g. one further active agent, such as an antiseptic, as far as they do not contradict the purpose of the present invention.

The following Examples illustrate the invention. The compounds are in free, i.e. neutral or base form unless specified otherwise.

### EXAMPLE 1

### Cream (Protective Hydrophobic Film)

| Component | Amount (g) |
|---|---|
| 33-Epichloro-33-desoxyascomycin | 1.00 |
| dimethicone | 5.00 |
| triglycerides, medium chain | 15.00 |
| oleyl alcohol | 10.00 |
| sodium cetylstearyl sulfate | 1.00 |
| cetyl alcohol | 4.00 |
| stearyl alcohol | 4.00 |
| glyceryl monostearate | 2.00 |
| benzyl alcohol | 1.00 |
| propylene glycol | 5.00 |
| citric acid | 0.05 |
| sodium hydroxide | * |
| water | ad 100.0 |

* amount required to adjust pH to 5.5

Preparation is according to conventional manufacturing procedures for an emulsion. The ascomycin derivative and dimethicone are added to the heated homogeneous oily phase which contains triglycerides medium chain, oleyl alcohol, sodium cetylstearyl sulfate, cetyl alcohol, stearyl alcohol and glyceryl monostearate. In parallel, the water phase containing the remaining ingredients is heated at the same temperature as the oily phase. The oily phase is added to the water phase and homogeneisation is performed. The resultant cream is cooled to room temperature.

### EXAMPLE 2

### Cream (with a Humectant)

The composition is as for Example 1, whereby dimethicone 5.00 g is replaced with glycerol 3.00 g, which for preparation is included in the water phase in place of the oily phase.

### EXAMPLE 3

### Cream (Semi-occlusive)

The composition is as for Example 1, whereby dimethicone 5.00 g is replaced with isostearyl isostearate 4.00 g.

### EXAMPLE 4

### Ointment (Protective Hydrophobic Film)

| Component | Amount (g) |
|---|---|
| 33-Epichloro-33-desoxyascomycin | 1.00 |
| dimethicone | 5.00 |
| oleyl alcohol | 10.00 |
| hexylene glycol | 10.00 |
| microcrystalline wax | 5.00 |
| white vaseline | ad 100.0 |

Preparation is according to conventional manufacturing procedures. The ascomycin is added to the heated homogeneous oily phase which contains dimethicone and the remaining ingredients. After homogeneisation the resultant ointment is cooled to room temperature.

### EXAMPLE 5

### Solution (Protective Hydrophobic Film)

| Component | Amount (g) |
|---|---|
| 33-Epichloro-33-desoxyascomycin | 1.00 |
| dimethicone | 5.00 |
| triglycerides, medium chain | 10.00 |
| oleyl alcohol | 10.00 |
| liquid paraffin | ad 100.0 |

Preparation is according to conventional manufacturing procedures and is as described under Example 4.

### EXAMPLE 6

### Liciuid Emulsion (with a Humectant)

| Component | Amount (g) |
|---|---|
| 33-Epichloro-33-desoxyascomycin | 1.00 |
| glycerol | 3.00 |
| triglycerides, medium chain | 15.00 |
| oleyl alcohol | 10.00 |
| glyceryl monooleate | 2.00 |
| Tween 80 | 4.00 |
| benzyl alcohol | 1.00 |
| propylene glycol | 5.00 |
| citric acid | 0.05 |
| sodium hydroxide | * |
| water | ad 100.0 |

* amount required to adjust pH to 5.5

Preparation is according to conventional manufacturing procedures. The ascomycin is added to the heated homogeneous oily phase which contains triglycerides medium chain, oleyl alcohol and glyceryl monooleate. In parallel, the water phase containing glycerol and the remaining ingredients is heated at the same temperature as the oily phase. The oily phase is added to the water phase and homogeneisation is performed. The resultant emulsion is cooled to room temperature.

### EXAMPLE 7

### Liquid Emulsion (Semi-occlusive)

As for Example 6, whereby glycerol 3.00 g is replaced with isostearyl isostearate 4.00 g, which for preparation is included in the oily phase in place of the water phase.

1. A pharmaceutical composition comprsing 33-epichloro-33-desoxyascomycin in combination or association with an emollient selected from the group consisting of dimethicone, glycerol and isostearylstearate together with at least one pharmaceutically acceptable diluent or carrier.

2. A pharmaceutical composition of claim 1 wherein the emollient is present in an amount from about 10% to about 5000% w/w of the amount of 33-epichloro-33-desoxyascomycin.

3. A method of treatment of a dermatological or mucosal disease in a subject suffering from such a disease comprising co-administering synergistically effective amounts of a composition of claim 1.

4. A process for the preparation of a composition of claims 1 comprising mixing 33-epichloro-33-desoxyascomycin and an emollient selected from the group consisting of dimethicone, glycerol and isostearylstearate in combination or association with at least one pharmaceutically acceptable diluent or carrier.

5. A kit of parts comprising 33-epichloro-33-desoxyascomycin and an emollient selected from the group consisting of dimethicone, glycerol and isostearylstearate in separate unit dosage forms, together with instructions for use.
